(19) United States
(12) Patent Application Publication
(10) Pub. No.: US 2023/0127357 A1
(43) Pub. Date: Apr. 27, 2023

(54) MIXED MODE CATIONIC EXCHANGE CHROMATOGRAPHY LIGANDS BASED ON SUBSTITUTED 2-BENZAMIDO ACETIC ACID STRUCTURES

(71) Applicant: BIO-RAD LABORATORIES, INC., HERCULES, CA (US)

(72) Inventors: CHRISTOPHER BELISLE, WALNUT CREEK, CA (US); JIALI LIAO, SAN RAMON, CA (US); CALLISTA KATRINA ASUNCION, LAFAYETTE, CA (US); EDWIN LOZANO, BERKELEY, CA (US)

(21) Appl. No.: 18/046,967

(22) Filed: Oct. 17, 2022

Related U.S. Application Data
(60) Provisional application No. 63/255,985, filed on Oct. 15, 2021.

(51) Int. Cl. C07K 1/16

(52) U.S. Cl. CPC C07K 1/165

(57) ABSTRACT

The subject invention pertains to mixed mode chromatography ligands and chromatography matrices suitable for the purification of proteins from biological sources or biological samples. Methods of making chromatography matrices comprising the disclosed ligands and using the disclosed chromatography matrices are also provided.

### CROSS-REFERENCE TO RELATED APPLICATION

This application claims the benefit of U.S. Provisional Application Ser. No. 63/255,985, filed Oct. 15, 2021, the disclosure of which is hereby incorporated by reference in its entirety, including all figures, tables and amino acid or nucleic acid sequences.

### FIELD OF THE INVENTION

Materials and methods for separating immunoglobulins or other proteins from source liquids, for purposes of purification or isolation, utilizing chromatographic separation techniques are provided. Methods of preparing chromatographic materials suitable for use in such techniques are also provided.

### BACKGROUND

The separation of proteins, such as immunoglobulins or other therapeutic biological agents, from source liquids, such as mammalian bodily fluids or cell culture harvest or supernatants, is of significant commercial interest and value. Also of interest are preparations of proteins in a sufficiently concentrated or purified form for diagnostic, laboratory, and therapeutic uses. However, the purification of proteins often suffers from factors such as low yield, the use of costly separation media (chromatography media), the leaching of separation media (for example, chromatography ligands) into the product, and concerns for the safe disposal of extraneous materials used in the extraction process. The present invention seeks to address at least some of these issues.

### BRIEF SUMMARY

This disclosure provides mixed mode chromatography ligands and chromatography matrices suitable for the purification of proteins from biological sources or samples. Methods of making chromatography matrices and using the disclosed chromatography ligands are also provided.

### BRIEF DESCRIPTION OF THE DRAWINGS

FIG. 1. Structures for chromatography ligand Nuvia cPrime.

FIG. 2. Elution profile for BL431.

FIG. 3. Elution profile for BL432.

FIG. 4. Elution profile for BL433.

FIG. 5. Elution profile for BL434.

FIG. 6. Elution profile for BL435.

FIG. 7. Elution profile for BL436.

FIG. 8. Elution profile for BL438.

FIG. 9. Elution profile for BL439.

FIG. 10. Elution profiled for BL441.

FIG. 11. Elution profiled for BL442.

FIG. 12. Elution profile for Nuvia cPrime.

FIGS. 13 (myoglobulin), 14 (ribonuclease A), and 15 (cytochrome c) provide a summary of the salt concentration (M) required to elute the tested proteins at pH 7, 6.5, 6, and 5.

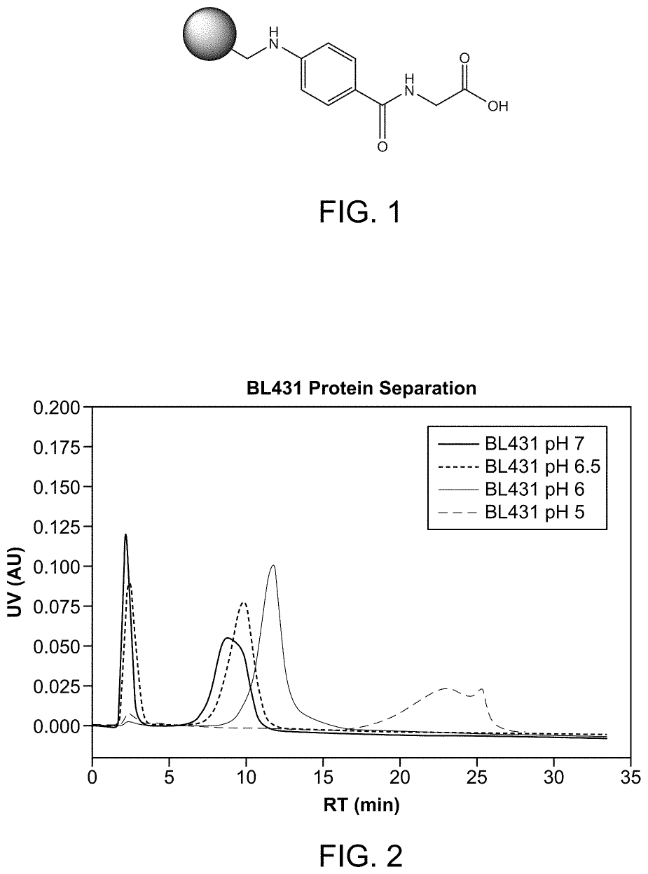

### DETAILED DISCLOSURE OF THE INVENTION

Unless otherwise stated, the following terms used in this application, including the specification and claims, have the definitions given below. As used in this specification and the appended claims, the singular forms “a”, “an”, and “the” include plural referents unless the content clearly dictates otherwise. Definition of standard chemistry terms can be found in reference works, including Carey and Sundberg (2007) “Advanced Organic Chemistry 5th Ed.” Vols. A and B, Springer Science+Business Media LLC, New York. The practice of the present invention will employ, unless otherwise indicated, conventional methods of synthetic organic chemistry, mass spectroscopy, preparative and analytical methods of chromatography, protein chemistry, biochemistry, recombinant DNA techniques and pharmacology.

The terms “biological sample(s)”, “source solution(s)”, or “source liquid(s)” refer to any composition containing a target molecule of biological origin (a “biomolecule”) that is desired to be purified. Non-limiting examples of target molecules include: antibodies, enzymes, growth regulators, clotting factors, transcription factors and phosphoproteins. In some embodiments, the target molecule (biomolecule) to be purified is an antibody or a non-antibody protein. Non-limiting examples of biological samples include serum samples from individuals or cell culture supernatants (e.g., clarified cell culture supernatants). With respect to the purification of biomolecules, such as antibodies, any biological sample that contains the target biomolecule can be used. Non-limiting examples of a source solution or source liquid include unpurified or partially purified antibodies from natural, synthetic, or recombinant sources. Unpurified antibody preparations (source solutions) can come from various sources including, but not limited to, plasma, serum, ascites, milk, plant extracts, bacterial lysates, yeast lysates, or conditioned cell culture media. Partially purified antibody preparations can come from unpurified preparations that have been processed by at least one chromatography, precipitation, other fractionation step, or any combination of the foregoing. In some embodiments, the antibodies have not been purified by protein A affinity prior to purification. Other embodiments utilize antibody preparations that have undergone a preliminary affinity purification step utilizing protein A or protein G.

“Antibody” refers to an immunoglobulin, composite (e.g., fusion protein), or fragmentary form thereof. The term includes but is not limited to polyclonal or monoclonal antibodies of the classes IgA, IgD, IgE, IgG, and IgM, derived from human or other mammalian cell lines, including natural or genetically modified forms such as humanized, human, single-chain, chimeric, synthetic, recombinant, hybrid, mutated, grafted, and in vitro generated antibodies. “Antibody” also includes composite forms including but not limited to fusion proteins containing an immunoglobulin moiety. “Antibody” also includes antibody fragments such as Fab, F(ab′)₂, Fv, scFv, Fd, dAb, Fc, whether or not they retain antigen-binding function.

The term “protein” refers to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers (e.g., recombinant proteins).

“Bind-elute mode” refers to an operational approach to chromatography in which the buffer conditions are established so that target molecules and, optionally undesired contaminants, bind to the ligand when the sample is applied to the ligand. Fractionation of the target can be achieved subsequently by changing the conditions such that the target is eluted from the support. In some embodiments, contaminants remain bound following target elution. In some embodiments, contaminants either flow-through or are bound and eluted before elution of the target.

“Flow-through mode” refers to an operational approach to chromatography in which the buffer conditions are established so that the target molecule to be purified flows through the chromatography support comprising the ligand, while at least some sample contaminants are selectively retained, thus achieving their removal from the sample.

The terms “matrix” or “support matrix” can be used interchangeably. In various embodiments, the matrix can be particles, a membrane or a monolith, and by “monolith” is meant a single block, pellet, or slab of material. Particles when used as matrices can be spheres or beads, either smooth-surfaced or with a rough or textured surface. Many, and in some cases all, of the pores are through-pores, extending through the particles to serve as channels large enough to permit hydrodynamic flow or fast diffusion through the pores. When in the form of spheres or beads, the median particle diameter, where the term “diameter” refers to the longest exterior dimension of the particle, is preferably within the range of about 25 microns to about 150 microns. The spheres or beads can have pores of a median diameter of 0.5 micron or greater, optionally with substantially no pores of less than 0.1 micron in diameter. In certain embodiments of the invention, the median pore diameter ranges from about 0.5 micron to about 2.0 microns. The pore volume can vary, although in many embodiments, the pore volume will range from about 0.5 to about 2.0 cc/g. Disclosures of matrices meeting the descriptions in this paragraph and the processes by which they are made are found in Hjerten et al., U.S. Pat. No. 5,645,717, Liao et al., U.S. Pat. No. 5,647,979, Liao et al., U.S. Pat. No. 5,935,429, and Liao et al., U.S. Pat. No. 6,423,666. Examples of monomers that can be polymerized to achieve useful matrices are vinyl acetate, vinyl propylamine, acrylic acid, methacrylate, butyl acrylate, acrylamide, methacrylamide, vinyl pyrrolidone (vinyl pyrrolidinone), with functional groups in some cases. Crosslinking agents are also of use in many embodiments, and when present will generally constitute a mole ratio of from about 0.1 to about 0.7 relative to total monomer. Examples of crosslinking agents are dihydroxyethylenebisacrylamide, diallyltartardiamide, triallyl citric triamide, ethylene diacrylate, bisacrylylcystamine, N,N′-methylenebisacrylamide, and piperazine diacrylamide.

The chromatography ligands are linked to the chromatography matrix via a linker to form a “chromatography resin” or “chromatography matrix”. Linkage of the chromatography ligand to the matrix will depend on the specific matrix used and the chemical group to be linked to the matrix. Ligands can be linked to the matrix by performing a reaction between the ligand, for example and amine group, and a functional group on the matrix, for example, an aldehyde or diol group. For matrices that do not have a suitable functional group, the matrix is reacted with a suitable activating reagent to create a suitable functional group to which the chromatography ligand can be attached.

For purposes of the formation of a linkage with the chromatography ligand, the inclusion of monomers with vicinal diols attached to the matrix is useful. One monomer example is allyloxy propandiol (3-allyloxy-1,2-propanediol). Vicinal diol monomers can be used with other monomers to prepare copolymers. The diol group density in the polymers produced from diol-containing monomers can vary widely, such as for example densities within a range of from about 100 to 1,000 μmol/mL (i.e., micromoles of diol per milliliter of packed beads), and in many cases a range of from about 200 to 300 μmol/mL. An example of a matrix that meets this description and is commercially available is UNOsphere™ Diol (Bio-Rad Laboratories, Inc., Hercules, Calif., USA). To couple a pendant amine-containing ligand to a matrix with exposed vicinal diols, the diols can be oxidized to aldehyde groups, and the aldehyde groups can then be coupled to amine groups to form secondary amino linkages, all by conventional chemistry techniques well known in the art. In some embodiments, the matrix comprises a diol, which is converted to an aldehyde, e.g., by conversion with NaIO₄. The primary amine of the ligand can be linked to an aldehyde on the matrix by a reductive amination reaction by the scheme provided in Example 1.

As used herein, the term “linker” refers to a molecule having 1-10 carbon atoms, preferably an alkyl group. The linker has a neutral charge and can include cyclic groups. The linker links the chromatographic ligand to the chromatography matrix. As used herein, the term “alkyl” refers to a straight or branched, saturated, aliphatic radical having between 1-10 carbon atoms. For example, C₁-C₆ alkyl includes, but is not limited to, methyl, ethyl, propyl, isopropyl, butyl, isobutyl, sec-butyl, tert-butyl, pentyl, isopentyl, and/or hexyl. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two chemical groups together. This disclosure provides a variety of chromatography ligands, described in Table 1. Table 1 provides the ligand structure as well as an exemplary ligand-matrix structure. As would be apparent to those skilled in the art, the linker attaching the ligand to the solid support (matrix) can be an alkyl group between 1 and 10 carbons in length, preferably between 1 and 5 carbons in length, or 1 to 3 carbons in length.

As used herein, the term “about” or “approximately” means within an acceptable error range for the particular value as determined by one of ordinary skill in the art, which will depend in part on how the value is measured or determined, i.e., the limitations of the measurement system. In the context of reagent and/or analyte concentrations, the term “about” or “approximately” can mean a range of around a given value of 0-20%, 0 to 10%, 0 to 5%, or 0-1% of a given value (e.g., ±20%, ±10%, ±5% or ±1% of a given value). In the context of pH measurements, the terms “about” or “approximately” permit a variation of ±0.1 or ±0.2 unit from a stated value.

This disclosure provides a number of novel ligands suitable for use in mixed mode cationic exchange chromatography (MM CEX). The ligands have the general structure:

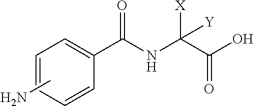

where X and Y can be the same or different and are independently selected from hydrogen a substituted or unsubstituted C₁-C₁₀ alkyl, a substituted or unsubstituted cycloalkyl, a substituted or unsubstituted C₁-C₁₀ alkene, or substituted or unsubstituted C₁-C₁₀ alkyne, with the proviso that both X and Y cannot be hydrogen. The alkyl, alkene or alkyne can be substituted by one, two or three radicals independently selected from C₁-C₁₀ alkyl, a carboxylic acid group, a carbonyl group, a benzyl group, a phenol group, an amine group, an indole group, a guanidino group, an imidazole group, a hydroxyl group, a thiol group, and a thiomethyl group. In some embodiments, X can form a cyclic structure with the adjacent amide (for example, a pyrrolidine) and Y is a hydrogen. In other embodiments, both X and Y are unsubstituted C₁-C₁₀ alkyl groups which can be the same or different in length and, if the same in length, can independently be linear or branched. In some embodiments, X and Y are the same or are different and are methyl or ethyl groups). The amine group can be at the ortho, para or meta position.

In some embodiments, Y is H and X is selected from:

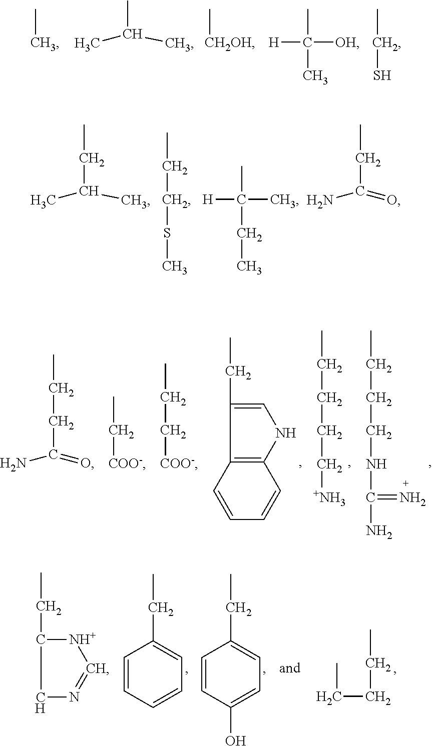

which forms a pyrrolidine with the adjacent nitrogen atom; or X and Y can be the same or different and are, independently, unsubstituted C₁-C₁₀ alkyl groups. which can be the same or different in length and, if the same in length, can independently be linear or branched. In some embodiments, X and Y are the same or are different and are methyl or ethyl groups).

In specific embodiments, These ligands are designated as, BL431, BL432, BL433, BL434, BL435, BL436, BL438, BL439, BL441, and BL442. The structures of these ligands is provided in Table 1.

TABLE 1

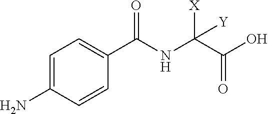

| | | Ligand | | |
|---|---|---|---|---|
| X | Y | Designation | Ligant Structure | Ligand Attached to Matrix |
| H | H | cPrime | | |
| COOH | H | BL431 | 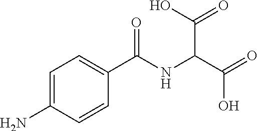 [SMILES: Nc1ccc(C(=O)NC(C(=O)O)C(=O)O)cc1] | 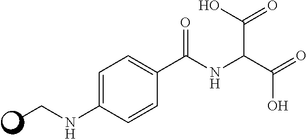 [SMILES: CCNc1ccc(C(=O)NC(C(=O)O)C(=O)O)cc1] |
| CH₂OH | H | BL432 | 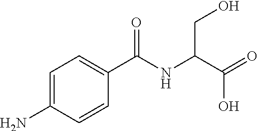 [SMILES: Nc1ccc(C(=O)NC(CO)C(=O)O)cc1] | 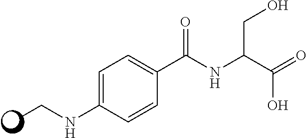 [SMILES: CCNc1ccc(C(=O)NC(CO)C(=O)O)cc1] |
| CH₃ | H | BL433 | 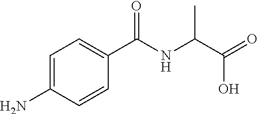 | 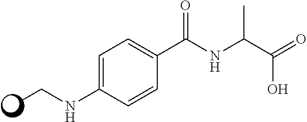 |
| CH₂Ph | H | BL434 | 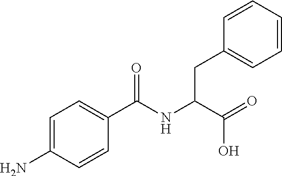 [SMILES: Nc1ccc(C(=O)NC(Cc2ccccc2)C(=O)O)cc1] | 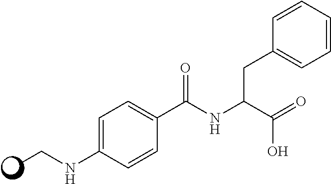 [SMILES: CCNc1ccc(C(=O)NC(Cc2ccccc2)C(=O)O)cc1] |
| CH₂CH₂COOH | H | BL436 | 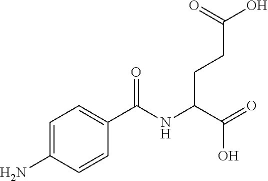 [SMILES: Nc1ccc(C(=O)NC(CCC(=O)O)C(=O)O)cc1] | 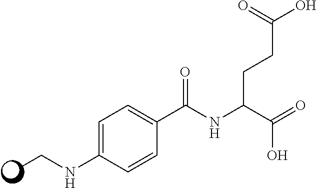 [SMILES: CCNc1ccc(C(=O)NC(CCC(=O)O)C(=O)O)cc1] |
| CH₂-Indole | H | BL435 | 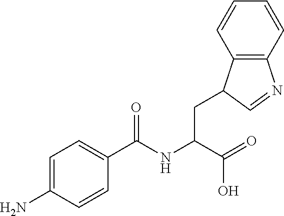 [SMILES: Nc1ccc(C(=O)NC(CC2C=Nc3ccccc32)C(=O)O)cc1] | 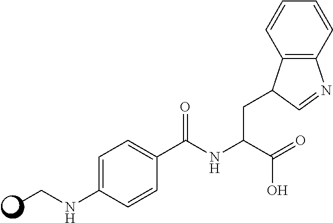 [SMILES: CCNc1ccc(C(=O)NC(CC2C=Nc3ccccc32)C(=O)O)cc1] |
| Pyrole | H | BL438 | 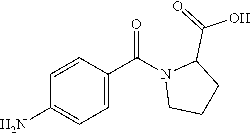 [SMILES: Nc1ccc(C(=O)N2CCCC2C(=O)O)cc1] | 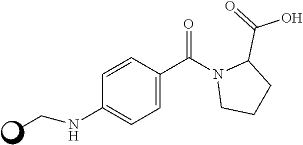 [SMILES: CCNc1ccc(C(=O)N2CCCC2C(=O)O)cc1] |
| Guanidine | H | BL439 | 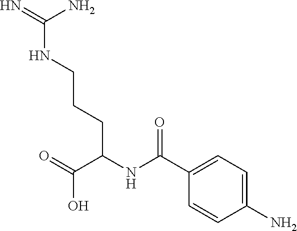 [SMILES: N=C(N)NCCCC(NC(=O)c1ccc(N)cc1)C(=O)O] | 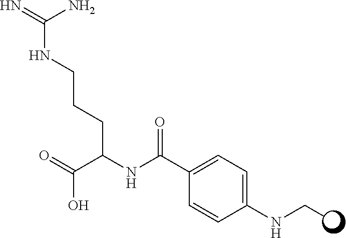 [SMILES: CCNc1ccc(C(=O)NC(CCCNC(=N)N)C(=O)O)cc1] |
| Branched | H | BL442 | 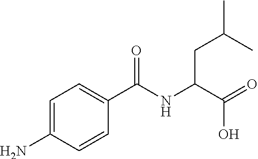 [SMILES: CC(C)CC(NC(=O)c1ccc(N)cc1)C(=O)O] | 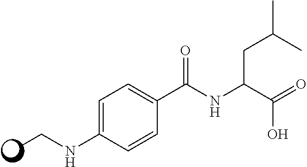 [SMILES: CCNc1ccc(C(=O)NC(CC(C)C)C(=O)O)cc1] |
| —CH₃ | —CH₃ | BL441 | 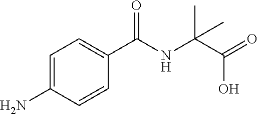 | 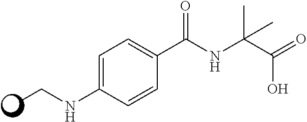 |

The disclosed ligands can be synthesized by standard chemical reactions. In some embodiments, amino acids (pure D-amino acid, pure L-amino acid, or a racemic mixtures of an amino acid) amino acids can be reacted with aminobenzoic acid to form a ligand in accordance with this disclosure. These ligands can then be immobilized on a solid support to form a chromatography resin. As would be apparent, the chromatography resin can, in some cases, be a chiral resin. Additionally, the amine functional group associated with the benzoic acid can be provided at the ortho, meta, or para position.

The disclosure also provides a mixed-mode chromatography medium having the formula:

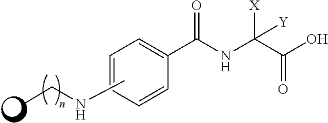

wherein:
the sphere is a solid support;
n is 1-10; and
X and Y can be the same or different and are independently selected from hydrogen, a substituted or unsubstituted C₁-C₁₀ alkyl, a substituted or unsubstituted C₁-C₁₀ alkene, or a substituted or unsubstituted C₁-C₁₀ alkyne, with the proviso that both X and Y cannot be a hydrogen and the nitrogen group coupled to the solid support can be at the ortho, para, or meta position.

In some embodiments, X and/or Y can be substituted with one, two, or three radicals independently selected from C₁-C₁₀ alkyl, a carboxylic acid group, a carbonyl group, a benzyl group, a phenol group, an amine group, an indole group, a guanidino group, an imidazole group, a hydroxyl group, a thiol group, and a thiomethyl group or X forms a pyrrolidine with the adjacent nitrogen atom. In some embodiments Y is hydrogen and X is selected from the group consisting of:

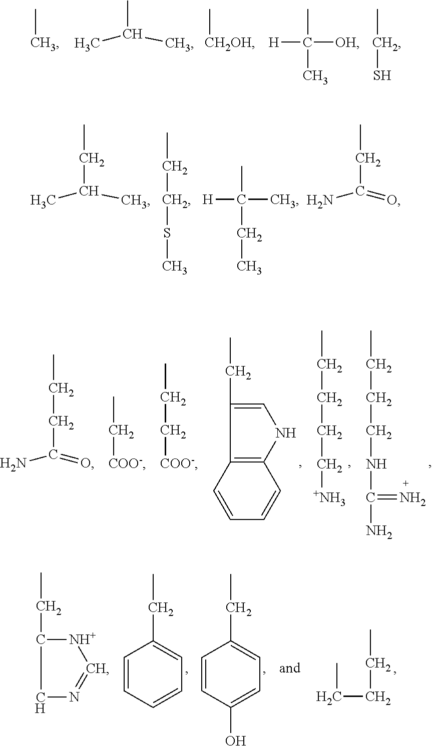

which forms a pyrrolidine with the adjacent nitrogen atom; or

X and Y can be the same or different and are, independently, unsubstituted C₁-C₁₀ alkyl groups. In other embodiments, both X and Y are unsubstituted C₁-C₁₀ alkyl groups which can be the same or different in length and, if the same in length, can independently be linear or branched. In some embodiments, X and Y are the same or are different and are methyl or ethyl groups).

Protein purification utilizing a chromatography resin in accordance with the present invention can be achieved by conventional means known to those of skill in the art. Examples of proteins include but are not limited to antibodies, enzymes, growth regulators, clotting factors, transcription factors and phosphoproteins. In many such conventional procedures, the chromatography resin prior to use is equilibrated with a buffer at the pH that will be used for the binding of the target protein (e.g., an antibody or a non-antibody protein). Equilibration can be done with respect to all features that will affect the binding environment, including ionic strength and conductivity when appropriate.

In some embodiments, the chromatography resins described herein can be used in “bind-elute” mode to purify a target protein from a biological sample. In some embodiments, following binding of the target protein to the chromatography resin, a change in pH can be used to elute the target protein.

In some embodiments, once the chromatography resin is equilibrated, a sample containing the target protein (e.g., a biological sample) is loaded onto the chromatography resin. The sample is maintained at a pH of between about 4.5 and about 8 with an appropriate buffer, allowing the target protein to bind to the chromatography resin. Notably, it has been found that the mixed mode chromatography resins described herein function with solutions having salt concentrations in the range of salt concentrations of cell cultures (e.g., 50-300 mM, or about 100-150 mM). Thus, in some embodiments, the protein is loaded to the chromatography resin under such salt concentrations.

In some embodiments, the chromatography resin is then washed with a wash buffer, optionally at the same pH as that of the loading step, to remove any proteins that may have been present in the source liquid. The bound target protein (e.g., antibody or non-antibody protein, as desired) can be subsequently eluted. In some embodiments, the protein is then eluted with an elution buffer at a pH above about 4.5, about 5.0, about 6.0, or about 7.0. Illustrative pH ranges, as cited above, are a pH of about 4.5 to about 8 for the binding and washing steps, and pH of about 4.5 to about 8, about 5.0 to about 8.0, about 6.0 to about 8.0, or about 7.0 to about 8.0 for the elution step. In certain embodiments, the binding and washing steps are performed with the inclusion of a salt in the sample and wash liquids. Examples of salts that can be used for this purpose are alkali metal and alkaline earth metal halides, notably sodium and potassium halides, and as a specific example sodium chloride. The concentration of the salt can vary; in most cases, an appropriate concentration will be one within the range of about 10 mM to about 1.5 M. As will be seen in the working examples below, optimal elution conditions for some proteins will involve a buffer with a higher salt concentration than that of the binding buffer, and in other cases by a buffer with a lower salt concentration than that of the binding buffer. The optimal choice in any particular case is readily determined by routine experimentation.

The chromatography resin can be utilized in any conventional configuration, including packed columns and fluidized or expanded-bed columns, and by any conventional method, including batchwise modes for loading, washes, and elution, as well as continuous or flow-through modes. The use of a packed flow-through column is particularly convenient, both for preparative-scale extractions and analytical-scale extractions. A column may, thus, range in diameter from 1 cm to 1 m, and in height from 1 cm to 30 cm or more. In some embodiments, a flow-through column can contain a mixture of particles, each particle comprising one of the chromatography ligands disclosed herein. In other embodiments, one or more chromatography ligand can be immobilized on a solid support, such as a particle, membrane or monolith to provide a chromatography resin that provides a mixture of chromatography ligands disposed on the solid support.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1—COUPLING OF LIGANDS TO A SOLID SUPPORT

### General Reaction Scheme

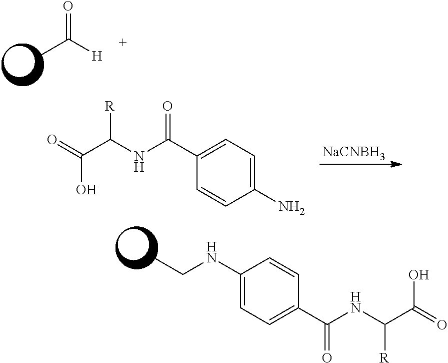

The general reaction scheme, illustrated above, utilizes a modified UNOsphere diol and ligand structure where “R” correspond to various amino acid and non-amino acid functionalities. These functionalities are illustrated below where R′ corresponds to the general ligand structure coupled to the solid support in the reaction scheme provided above. For BL438, the nitrogen atom is adjacent to the R group which is —CH₂—CH₂—CH₂— and forms a ring with the nitrogen atom.

### General Procedure for BL431, BL432, BL433, BL436, BL438, BL441, BL442

Ligand (˜1.3-1.7 mol eq.) was dissolved in an equal volume of UNOsphere aldehyde (150-250 μmol/mL) in 1 M sodium acetate (pH 4.5-5.0) and mixed with vigorous stirring at 37° C. for 1 hour. 0.0124 mg of sodium cyanoborohydride was then added per mL UNOsphere aldehyde and stirred vigorously at 37° C. overnight. The UNOsphere-ligand chromatography matrix was then washed with 20 column volumes of water.

### General Procedure for BL434 and BL435

Ligand (˜1.3-1.7 mol eq.) was dissolved in an equal volume of UNOsphere aldehyde (150-250 μmol/mL) in 50% THF and 1 M sodium acetate (pH 4.5-5.0) and mixed with vigorous stirring at 37° C. for 1 hour. 0.0124 mg of sodium cyanoborohydride was then added per mL UNOsphere aldehyde and stirred vigorously at 37° C. overnight. The UNOsphere-ligand chromatography matrix was then washed with 20 column volumes of water.

### General Procedure for BL439

Ligand (˜1.3-1.7 mol eq.) was dissolved in an equal volume of UNOsphere aldehyde (150-250 μmol/mL) in 50% THF and water (pH 1.5-2) and mixed with vigorous stirring at 37° C. for 1 hour. 0.0124 mg of sodium cyanoborohydride was added per mL UNOsphere aldehyde and stirred vigorously at 37° C. overnight. The UNOsphere-ligand chromatography matrix was then washed with 20 column volumes of water.

### EXAMPLE 2—GENERAL PROCEDURE FOR PROTEIN SEPARATION

A 2.2 mL column was packed with each of the immobilized ligands. Each column was equilibrated with Buffer A (20 mM sodium phosphate buffer at pH 7, 6.5, 6, and 5). Column elution was performed using a 30 column volume linear gradient of Buffer A to Buffer B (20 mM sodium phosphate buffer at pH 7, 6.5, 6, and 5+1.5 M NaCl). Each column was loaded with 250 μL of Bio-Rad's Cation Protein Standard in Buffer A. This protein standard elutes in the order of myoglobin (RT1), ribonuclease a (RT2), and cytochrome-c (RT3).

Each immobilized ligand had a ligand density of 52-120 μmol/mL with pKa ranging from 4.5-5.8. For comparison, chromatography ligands were analyzed against Nuvia cPrime which had a ligand density and pKa of 120 μmol/mL and 4.0 respectively.

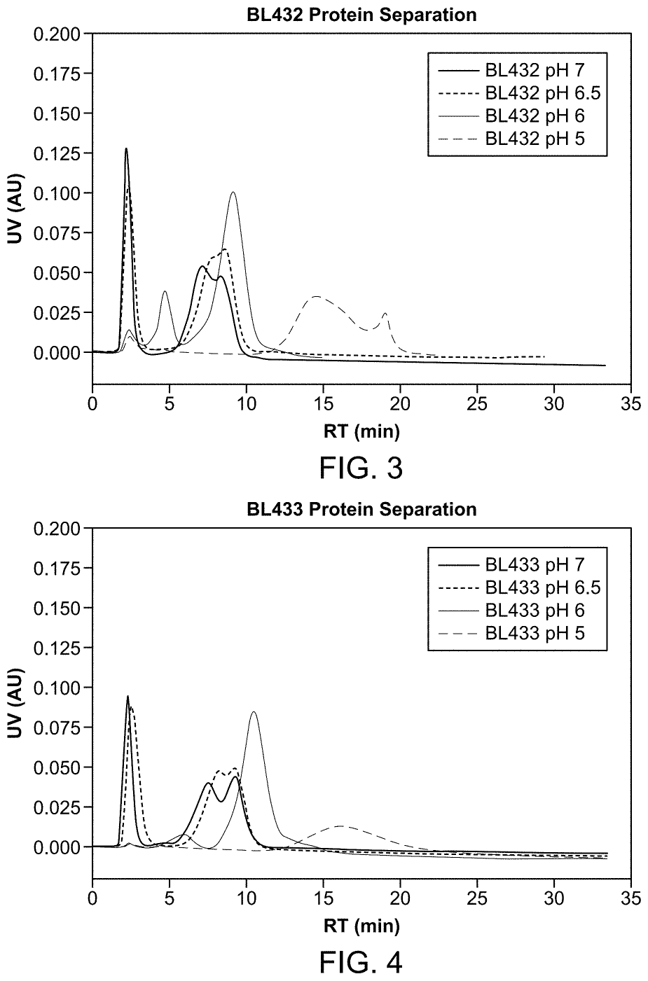

The chromatograph for BL431 is provided in FIG. 2 at pH 7, 6.5, 6, and 5. At pH 7, there's potential for separation amongst ribonuclease A and cytochrome-c. Although, as pH decreases both proteins seem to coelute. At pH 6 & pH 5 all three proteins coelute together. The chromatograph for BL432 is provided in FIG. 3. Good separation is observed at pH 7. At pH 6.5, the peaks seem to nearly coelute and all three proteins coelute at pH 6 and pH 5. The extra peak at 4.9 min is an impurity peak.

The chromatograph for BL433 is provided in FIG. 4. Excellent separation is observed at pH 7. Coelution begins to be observed at pH 6.5 and at pH 6 and pH 5, all three proteins coelute together.

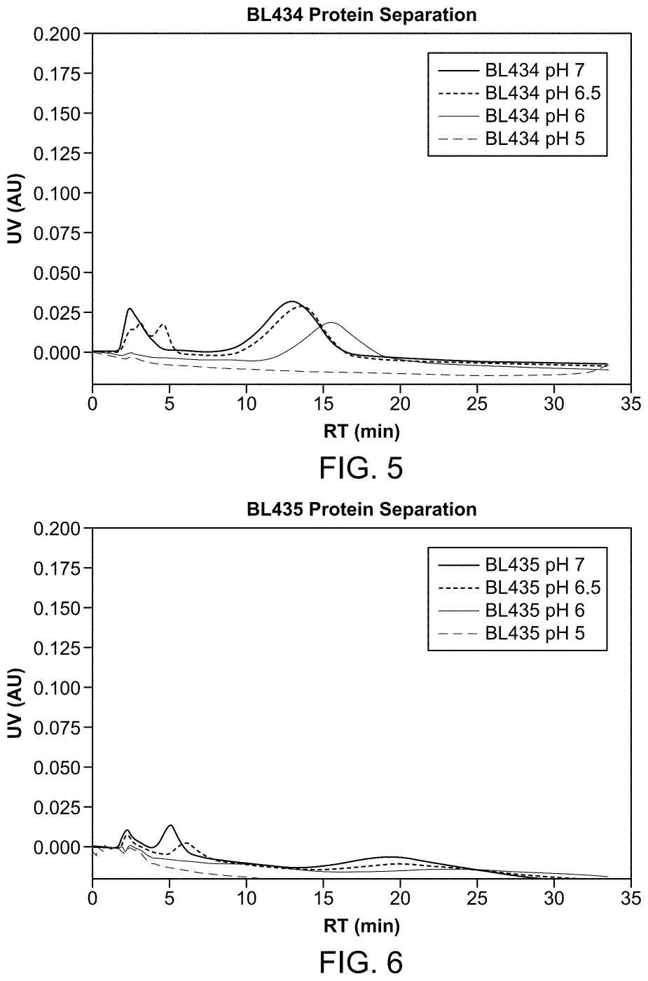

The chromatograph for BL434 is provided in FIG. 5. Under the tested elution conditions, the proteins all coelute at any given pH. At pH 5, all three proteins remain bound to the column.

The chromatograph for BL435 is provided in FIG. 6. Partial elution is observed at pH 7. As the pH decrease, hydrophobic interactions between the tested proteins and the chromatography ligand increase and the proteins remain bound to the column.

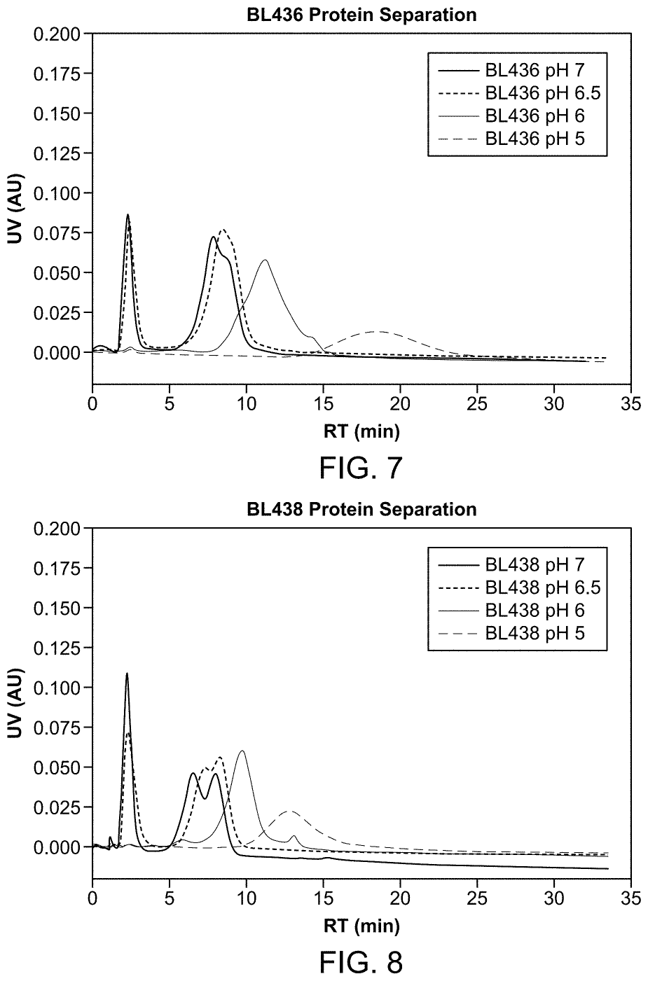

The chromatograph for BL436 is provided in FIG. 7. At pH 7, there is limited separation between ribonuclease A and cytochrome C. As the pH decreases, hydrophobic interactions increase for ribonuclease A and cytochrome C and the ligand. At pH 6 and pH 5, all three proteins coelute.

The chromatograph for BL438 is provided in FIG. 8. There is separation at pH 7 and some separation at 6.5. Coelution occurs at pH 6 and pH 5.

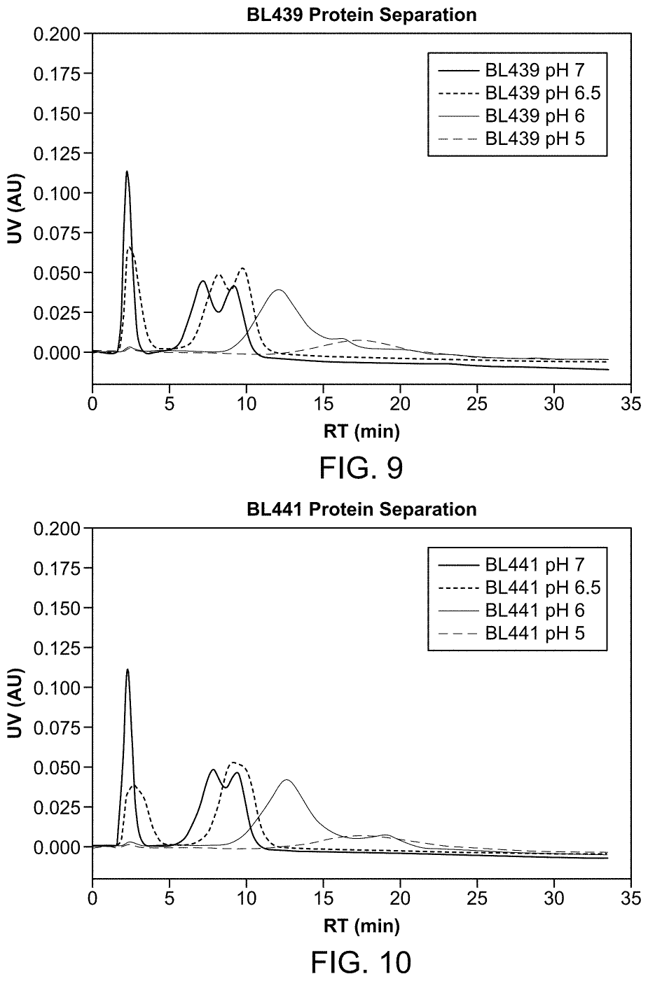

The chromatograph for BL439 is provided in FIG. 9. Good separation is observed at pH 7 and 6.5. Coelution occurred at pH 6 and pH 5.

The chromatograph for BL441 is provided in FIG. 10. There was some separation of the tested proteins at pH 7 but coelution of ribonuclease A and cytochrome c was observed at pH 6.5. At pH 6 and pH 5, all three proteins coeluted.

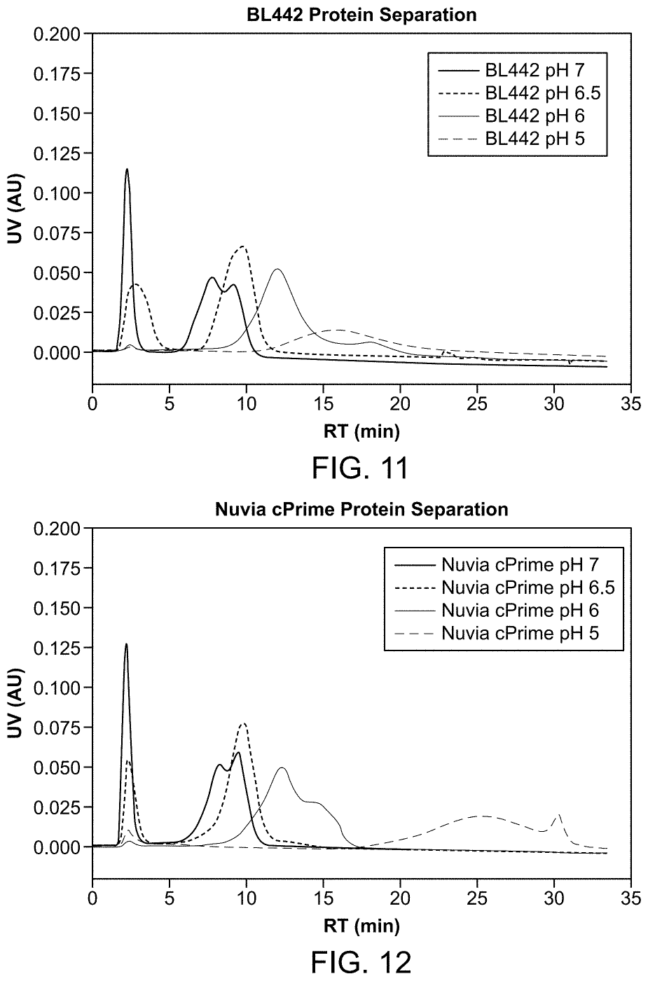

The chromatograph for BL442 is provided in FIG. 11. Good separation is observed at pH 7. There was some separation of the tested proteins at pH 7 but as pH decreased to pH 6.5, ribonuclease A and cytochrome c coeluted. At pH 6 and pH 5, all three proteins coeluted.

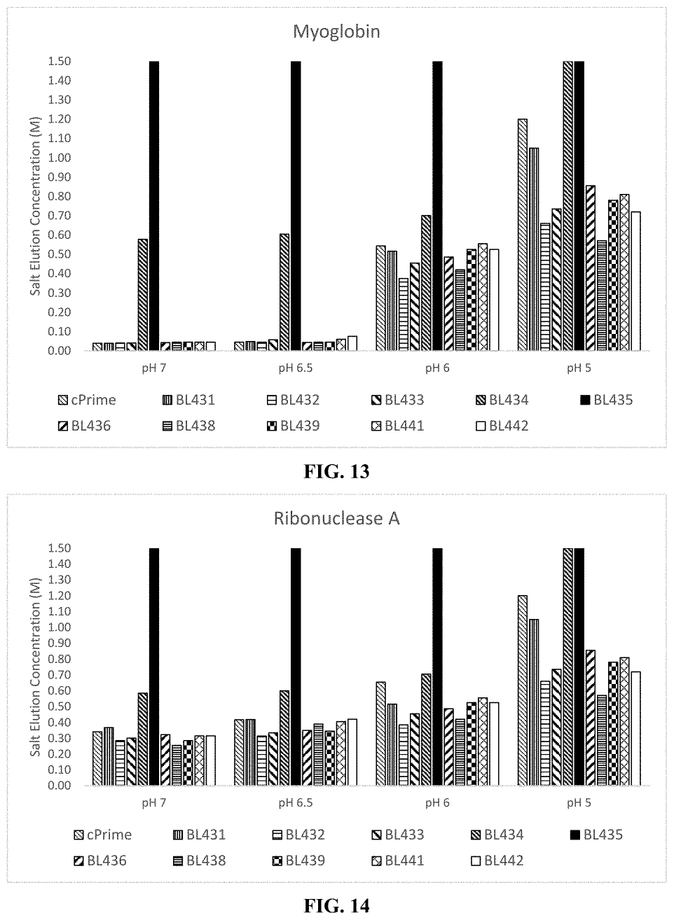

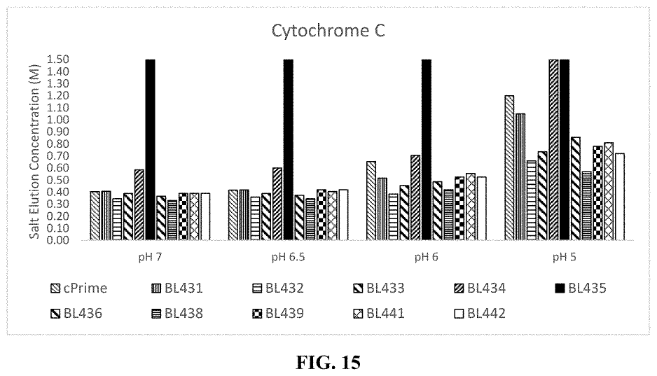

The chromatograph for Nuvia cPrime is provided in FIG. 12. Good separation is observed at pH 7. At pH 6.5, coelution occurs with ribonuclease A and cytochrome-c. At pH 6, all three proteins coelute together, although some separation is observed. At pH 5 there's a strong interaction and the protein's coelute over 12 min over 1 M NaCl (see also FIGS. 13-15).

FIGS. 13-15 provide a summary of the salt concentration (M) required to elute the tested proteins at pH 7, 6.5, 6, and 5 from the various ligands and Nuvia cPrime. Due to the hydrophobic nature of several of the disclosed ligands (e.g., BL435, BL436 and BL438), those ligands require more salt to elute proteins.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

We claim:

1. A method for purifying a protein from a source solution, said method comprising: (a) contacting said source solution with a mixed-mode chromatography medium comprising a mixed-mode chromatography ligand coupled to a solid support via its primary amine group; and (b) eluting said protein so bound from said solid support, said mixed-mode chromatography medium having the formula: 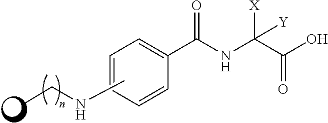
wherein:
the sphere is a solid support:
n is 1-10; and
X and Y can be the same or different and are independently selected from hydrogen, a substituted or unsubstituted C₁-C₁₀ alkyl, a substituted or unsubstituted C₁-C₁₀ alkene, or a substituted or unsubstituted C₁-C₁₀ alkyne, with the proviso that both X and Y cannot be a hydrogen.

2. The method according to claim 1, wherein X and/or Y is substituted with one, two, or three radicals independently selected from C₁-C₁₀ alkyl, a carboxylic acid group, a carbonyl group, a benzyl group, a phenol group, an amine group, an indole group, a guanidino group, an imidazole group, a hydroxyl group, a thiol group, and a thiomethyl group or X forms a pyrrolidine with the adjacent nitrogen atom.

3. The method according to claim 1, wherein Y is hydrogen and X is selected from the group consisting of: 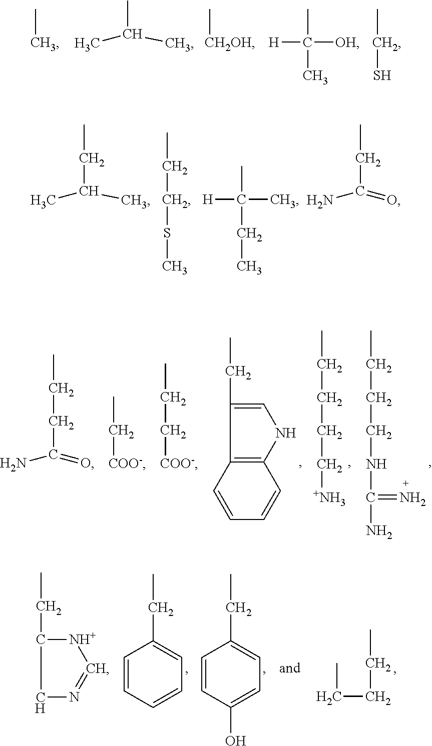
which forms a pyrrolidine with the adjacent nitrogen atom; or X and Y can be the same or different and are, independently, unsubstituted C₁-C₁₀ alkyl groups.

4. The method according to claim 1, wherein the ligand is a chiral ligand and the stereogenic carbon alpha to the amide group has a D-configuration or has an L-configuration.

5. A mixed-mode chromatography medium comprising a ligand coupled to a solid support, said ligand being selected from the group consisting of BL431, BL432, BL433, BL434, BL435, BL436, BL438, BL439, BL441, and BL442.

6. The mixed-mode chromatography medium according to claim 5, wherein the ligand is a chiral ligand and the stereogenic carbon alpha to the amide group has a D- configuration or has an L-configuration.

7. A method for manufacturing a mixed-mode chromatography medium, said method comprising: (a) oxidizing diol groups on diol-functionalized solid support, thereby converting said diol-functionalized solid support to an aldehyde-functionalized solid support; and (b) coupling amine-functionalized ligands to said aldehyde-functionalized solid support, said amine-functionalized ligands being selected from the group consisting of BL431, BL432, BL433, BL434, BL435, BL436, BL438, BL439, BL441, and BL442.

8. The method according to claim 7, wherein the ligand is a chiral ligand and the stereogenic carbon alpha to the amide group has a D-configuration or has an L-configuration.

9. A ligand of the structure 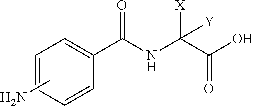
where X and Y can be the same or different and are independently selected from hydrogen, a substituted or unsubstituted C₁-C₁₀ alkyl, a substituted or unsubstituted C₁-C₁₀ alkene, or substituted or unsubstituted C₁-C₁₀ alkyne or X forms a cyclic structure with the adjacent amide, with the proviso that both X and Y cannot be a hydrogen.

10. The ligand according to claim 9, wherein the alkyl, alkene or alkyne is substituted by one, two or three radicals independently selected from C₁-C₁₀ alkyl, a carboxylic acid group, a carbonyl group, a benzyl group, a phenol group, an amine group, an indole group, a guanidino group, an imidazole group, a hydroxyl group, a thiol group, and a thiomethyl group or X forms a pyrrolidine ring with the adjacent amide.

11. The ligand according to claim 9, wherein Y is a hydrogen and X is selected from the group consisting of: 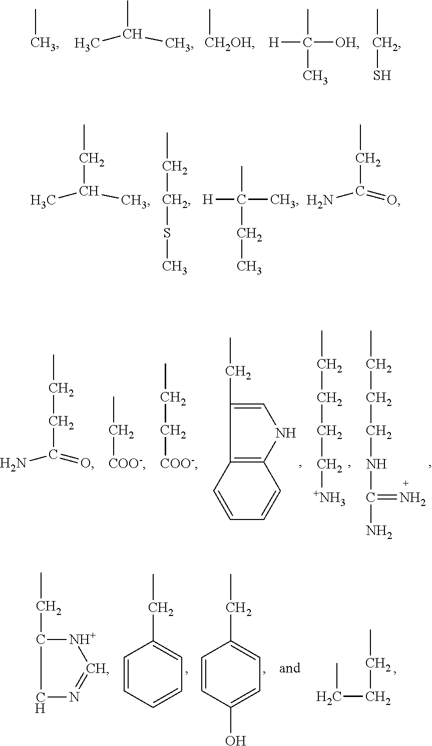
which forms a pyrrolidine with the adjacent nitrogen atom; or X and Y can be the same or different and are, independently, unsubstituted C₁-C₁₀ alkyl groups.

12. The ligand according to claim 9, wherein the ligand is a chiral ligand and the stereogenic carbon alpha to the amide group has a D-configuration or has an L-configuration.

13. The ligand according to claim 9, wherein the amine group is at an ortho, para, or meta position.

14. A chromatography resin comprising:
a support matrix, an optional linker and a ligand according claim 9, the ligand being coupled to the support matrix via the primary amine group of the ligand.

15. The chromatography resin according to claim 14, wherein the amine group is at an ortho, para, or meta position.

16. A mixed-mode chromatography medium comprising a solid support and a ligand and having the formula: 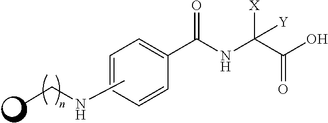
wherein:
the sphere is a solid support;
n is 1-10; and
X and Y can be the same or different and are independently selected from hydrogen, a substituted or unsubstituted C₁-C₁₀ alkyl, a substituted or unsubstituted C₁-C₁₀ alkene, or a substituted or unsubstituted C₁-C₁₀ alkyne, with the proviso that both X and Y cannot be a hydrogen and the nitrogen group can be at the ortho, para, or meta position.

17. The mixed-mode chromatography medium according to claim 16, wherein the ligand is a chiral ligand and the stereogenic carbon alpha to the amide group has a D-configuration or has an L-configuration.

18. The mixed-mode chromatography medium according to claim 16, wherein X and/or Y is/are substituted with one, two, or three radicals independently selected from C₁-C₁₀ alkyl, a carboxylic acid group, a carbonyl group, a benzyl group, a phenol group, an amine group, an indole group, a guanidino group, an imidazole group, a hydroxyl group, a thiol group, and a thiomethyl group or X forms a pyrrolidine with the adjacent nitrogen atom.

19. The mixed-mode chromatography medium according to claim 16, wherein Y is hydrogen and X is selected from the group consisting of: 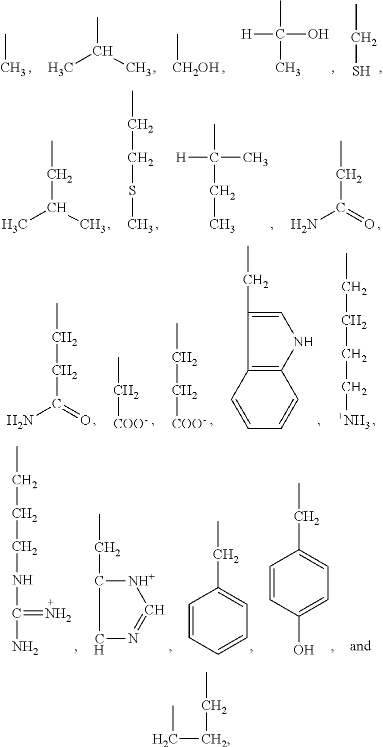
which forms a pyrrolidine with the adjacent nitrogen atom; or X and Y can be the same or different and are, independently, unsubstituted C₁-C₁₀ alkyl groups.

20. A method for manufacturing a mixed-mode chromatography medium, said method comprising: (a) oxidizing diol groups on diol-functionalized solid support, thereby converting said diol-functionalized solid support to an aldehyde-functionalized solid support; and (b) coupling amine-functionalized ligands according to claim 9 to said aldehyde-functionalized solid support.
